(19) United States
(12) Patent Application Publication
(10) Pub. No.: US 2022/0241455 A1
(43) Pub. Date: Aug. 4, 2022

(54) WOUND DRESSING

(71) Applicant: Nan Liu Enterprise Co., LTD., Kaohsiung City (TW)

(72) Inventors: Chin-San Huang, Kaohsiung City (TW); Ho-Chun Huang, Kaohsiung City (TW)

(21) Appl. No.: 17/168,138

(22) Filed: Feb. 4, 2021

(51) Int. Cl. A61L 15/44; A61L 15/42; A61L 15/40; A61L 15/24; A61L 15/26; A61L 15/22; A61F 13/00

(52) U.S. Cl. CPC A61L 15/44; A61L 15/425; A61L 15/40; A61L 15/24; A61L 15/26; A61L 2300/104; A61F 13/00042; A61F 13/00063; A61F 13/00012; A61F 13/00017; A61F 13/00029; A61L 15/225

(57) ABSTRACT

A wound dressing is provided, which includes a substrate and a plurality of silver particles. The substrate has a first surface and a second surface which are opposite to each other, and the substrate further includes a plurality of microstructures. A plurality of silver particles is embedded rather in the microstructures or in the gaps between the microstructures, between first surface and the second surface. The wound dressing has a moisture vapor transmission rate being about 4000-20000 g/(m²*24 hr).

### BACKGROUND OF THE INVENTION

### 1. Field of the Invention

The present invention relates to a wound dressing, and more particularly, to a wound dressing which may be applied on various traumas.

### 2. Description of the Prior Art

Wound dressing is a simple treatment material for reducing infection or stimulating cell repair. Recently, various designs of wound dressings have been developed and used in wound-care, in order to hasten the wound healing process, such as an antibacterial dressing, an extensible dressing, a breathable dressing, a water-retaining dressing and the like. However, the currently available wound dressings still have obvious drawbacks. For example, general wound dressings are poorly efficient in long-term healing, so that, which may lead to unnecessary inconvenience to patient suffering from wounds recovery that needs long-term healing period (such as diabetic ulcer or scald), which may require frequent change of wound dressing. On the other hand, additional bactericidal agents or antibacterial gradients may be used on some common wound dressings to avoid inflammation and infection. However, the additional bactericidal agents or antibacterial gradients may entail serious cytotoxicity, which may cause discomfort and aggravate the pain, thereby still being less useful to long-term use in wounds. Thus, there is still a crucial need to provide new design of wound dressing so as to meet the therapeutic product requirements.

### SUMMARY OF THE INVENTION

It is one of the primary objectives of the present invention to provide a wound dressing, in which a plurality of silver particles is embedded inside the microstructures or between gaps of the microstructures of the substrate, such that, the silver particles are not allowed to be freely released from the substrate during the infiltration or scouring by body fluid. Accordingly, the wound dressing may achieve the effects both on lower biological toxicity and long-term application.

To achieve the purpose described above, one embodiment of the present invention provide a wound dressing including a substrate and a plurality of silver particles. The substrate includes a first surface and a second surface opposite to each other, with the substrate including a plurality of microstructures. The plurality of the silver particles is embedded in gaps of the microstructures or embedded inside each of the microstructures, between the first surface and the second surface, wherein the moisture vapor transmission rate (MVTR) of the wound dressing is about 4000-20000 g/m²*24 hr.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram illustrating a cross-sectional view of a wound dressing according to a first embodiment of the present invention.

FIG. 2 is a schematic diagram illustrating a cross-sectional view of a wound dressing according to another embodiment of the present invention.

FIG. 3 is a schematic diagram illustrating a cross-sectional view of a wound dressing according to another embodiment of the present invention.

FIG. 4 is a schematic diagram illustrating a cross-sectional view of a wound dressing according to a second embodiment of the present invention.

FIG. 5 is a schematic diagram illustrating a cross-sectional view of a wound dressing according to a third embodiment of the present invention.

FIG. 6 is a schematic diagram illustrating a fixing structure of a wound dressing according to a preferably embodiment of the present invention.

FIG. 7 is a schematic diagram illustrating another fixing structure of a wound dressing according to another embodiment of the present invention.

FIG. 8 is a schematic diagram illustrating the application of a wound dressing.

### DETAILED DESCRIPTION

For better understanding of the presented disclosure, preferred embodiments will be described in detail. The preferred embodiments of the present invention are illustrated in the accompanying drawings with numbered elements.

In the present invention, the notion of “the formation of a first component over or on a second component” may be directed to embodiments in which the first and second components are formed in direct contact, and may also include embodiments in which additional components may be formed between the first and second components, such that the first and second components may not be indirect contact. In addition, the present invention may repeat reference numerals and/or letters in various examples. This repetition is for the purpose of simplicity and clarity and does not in itself dictate a relationship between the various embodiments and/or configurations discussed. Furthermore, spatially relative terms, such as “beneath,” “below,” “lower,” “over,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. For example, if the device in the figures is turned over by 180 degrees, elements described as “below” and/or “beneath” other elements or features would then be oriented “above” and/or “over” the other elements or features. The apparatus may be otherwise oriented (rotated 90 degrees or at other orientations) and the spatially relative descriptors used herein may likewise be interpreted accordingly.

It is understood that, although the terms first, second, third, etc. may be used herein to describe various elements, components, regions, layers and/or sections, these elements, components, regions, layers and/or sections should not be limited by these terms. These terms may be only used to distinguish one element, component, region, layer and/or section from another region, layer and/or section. Terms such as “first,” “second,” and other numerical terms when used herein do not imply a sequence or order unless clearly indicated by the context. Thus, a first element, component, region, layer and/or section discussed below could be termed a second element, component, region, layer and/or section without departing from the teachings of the embodiments.

As disclosed herein, the term “about” or “substantial” generally means within 20%, preferably within 10%, and more preferably within 5%, 3%, 2%, 1%, or 0.5% of a given value or range. Unless otherwise expressly specified, all of the numerical ranges, amounts, values and percentages disclosed herein should be understood as modified in all instances by the term “about” or “substantial”. Accordingly, unless indicated to the contrary, the numerical parameters set forth in the present invention and attached claims are approximations that can vary as desired.

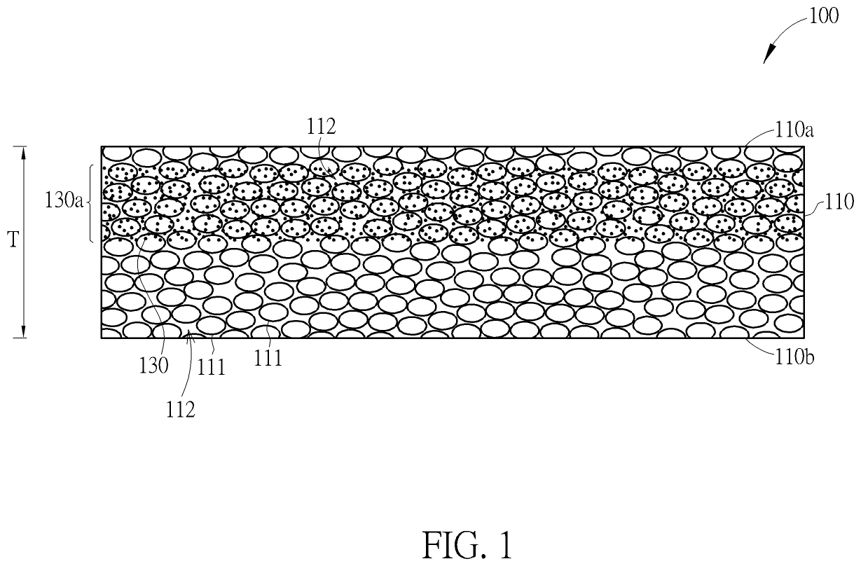

Please refer to FIG. 1, which illustrates a wound dressing 100 according to the first embodiment of the present invention. The wound dressing 100 includes a substrate 110, and a plurality of metal particle 130 embedded inside the substrate 100, wherein the metal particle for example includes antibacterial metal particles which may be selected from a group including silver (Ag), iron (Fe), nickel (Ni), copper (Cu), chromium (Cr), manganese (Mn), gold (Au), gallium (Ga), mercury (Hg), lead (Pb), aluminum (Al), zinc (Zn), bismuth (Bi), tin (Sn) and palladium (Pd), preferably includes silver particles but not limited thereto. The substrate 110 in detail may include a plurality of microstructures 111, the microstructures 111 are alternately arranged into a porous structure (not shown in the drawings), with each of the metal particles 130 being optionally embedded in the holes (or gaps) 112 of the porous structure and/or embedded in each of the microstructures 111, as shown in FIG. 1. Each of the microstructures 111 preferably includes a smooth surface, with a length of each microstructure 111 being about 35 (millimeter, mm) to 45 mm, preferably 38 mm, with a density of each microstructure 111 being about 1.6-2.0 g/10 km (g/10 km), preferably 1.7 g/10 km; and with a moisture vapor transmission rate of the microstructure 111 being about 4000-20,000 g/m²*24 hr, preferably being about 15,000 g/m²*24 hr. The moisture vapor transmission rate refers to the amount of water vapor per unit (g) penetrate through per unit area (m²) of the microstructures 111 during a period of time (24 hours). It is noted that FIG. 1 only illustrates the cross-sectional shape of the microstructure 111 along a cross-line direction (not shown in the drawings), without illustrating the overall appearance of the microstructures 111. People in the art should easily understand that the specific appearance, the arrangement, the shape and the size of the microstructures 111 may be different by the material selection of the substrate 110, and which is not limited to be what is shown in FIG. 1. Accordingly, the porous structure which is composed by the microstructures 111 may further include different forms. For example, in one embodiment, the substrate 110 may include a fiber material including a fiber sponge structure (not shown in the drawings) which is interwoven by a plurality of microfibers, with each of the metal particles 130 being embedded in the gaps between each microfiber and/or being embedded inside each microfiber. In another embodiment, the substrate 110 may include a polymer material including a network structure which is composed of a plurality of microporous polymers (not shown in the drawings), with each metal particle 130 being embedded inside the holes within the network structure and/or being embedded inside each microporous polymer, but not limited thereto.

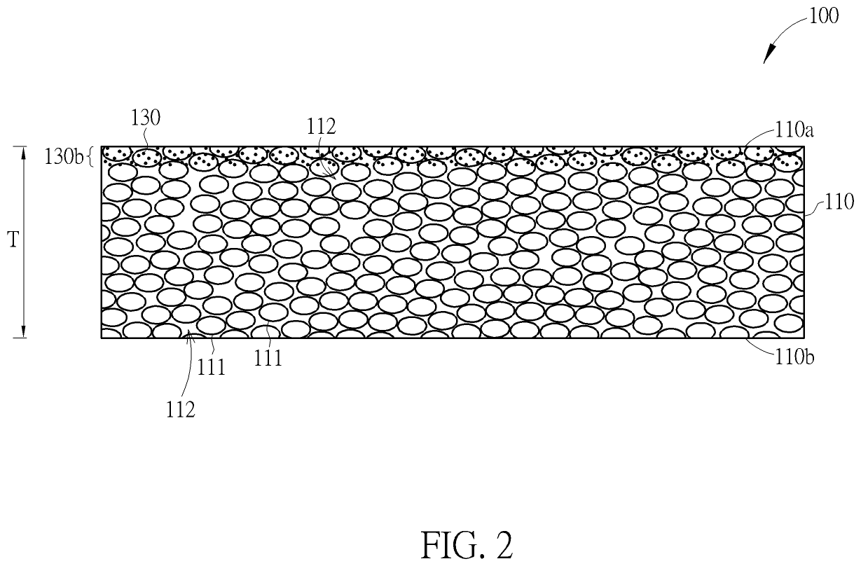

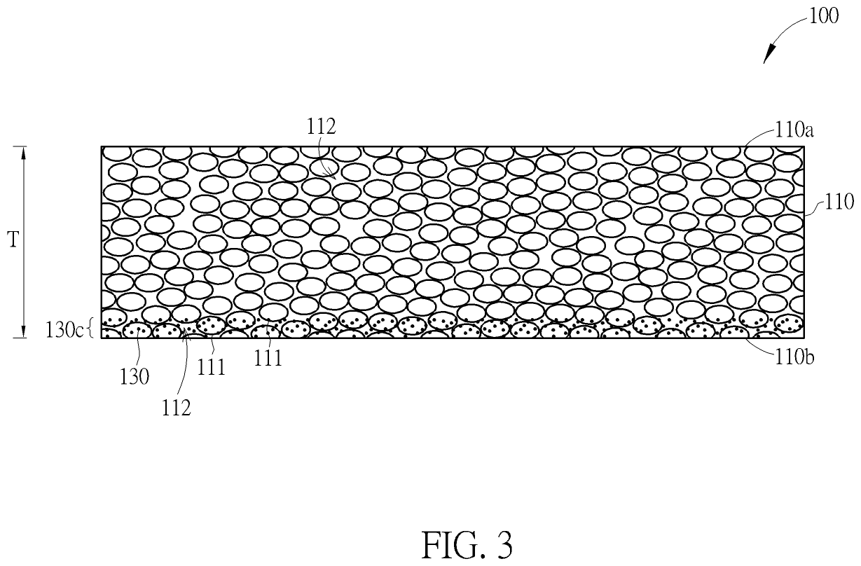

A weight ratio of the metal particles 130 related to the substrate 110 is about 10-250 mg metal particles 130 (10-250 mg/100 cm²) in per 100 square centimeters of the substrate 110, preferably is about 50-150 mg of metal particles 130 (50-150 mg/100 cm²), but is not limited thereto. It is noted that the metal particles 130 are uncharged and physically attached between and/or inside the microstructures 111 of the substrate 110, so that the metal particles 130 are difficult to be freely released from the substrate 110, thereby avoiding the metal particles 130 to be in direct contact with the wound opening to bring out toxic complications. As an example, a sputtering process may be performed through colliding a metal target by an inert gas (such as argon), thereby disposing the metal particles inside the substrate 110, but is not limited to. Preferably, while the sputtering process is performed, a magnetic force or gravity may be additionally applied, so that, the metal particles 130 generated thereby may be aggregated at a specific location or a specific region of the substrate. In the present embodiment, a concentration region 130a of the metal particles 130 may be disposed between two opposite surfaces (such as the first surface 110a and the second surface 110b), without directly exposing from the first surface 110a or the second surface 110b, as shown in FIG. 1. If the first surface 110a is disposed at upper side, the concentration region 130a of the metal particles 130 may extends from one tenth to one half of a thickness “T” of the substrate 110, as shown in FIG. 1. Accordingly, most of the metal particles 130 are embedded between one tenth of the thickness “T” of the substrate 110 and one half of the thickness “T” of the substrate 110, adjacent to the first surface 110a, but is not limited thereto. In another embodiment, a concentration region (not shown in the drawings) of the metal particles 130 may also optionally extend from one tenth to one third of the thickness “T” of the substrate 110, or only located at one tenth, one third or one half of the thickness “T” of the substrate 110. In addition, although the concentration region 130a of the metal particles 130 is exemplified by being disposed closer to the first surface 110a of the substrate 110 in the present embodiment, the practical embodiment is not limited thereto. In another embodiment, the concentration of the metal particles may also be disposed closer to the second surfaces 110b due to practical product requirements. Otherwise, as shown in FIGS. 2-3, the concentration regions 130b, 130c of the metal particles 130 may respectively extend from the first surface 110a or the second surface 110b of the substrate 100 to one tenth, one third or one half of the thickness “T” of the substrate 110. In other words, under different product requirements, a portion of the metal particles 130 may be embedded on the first surface 110a or the second surface 110b of the substrate, with the metal particles being directly exposed from the first surfaces 110a or the second surfaces 110b, and with the concentration region thereof further distributing from the first surface 110a or the second surface 110b to one half of the thickness “T” of the substrate 110, from the first surface 110a or the second surface 110b to one third of the thickness “T” of the substrate 110, or from the first surface 110a or the second surface 110b to one tenth of the thickness “T” of the substrate 110, and so on.

Precisely, if the substrate 110 includes the fiber material, such as a natural microfiber or a synthetic microfiber, wherein the natural microfibers may be selected from a group of cotton, wood, linen fiber, jute fiber, ramie fiber, shengma fiber, hemp fiber, rayon, modal, lyocell fiber and tencel cotton; and the synthetic microfiber may be selected from a group of natural or (semi) synthetic hydrophilic shortfibers including polyester (PET), polyamide 6 (PA6), polyamide 66 (PA66), nylon 6, nylon 66, polypropylene (PP), polyolefin, acetate fiber, polyethylene fiber, polyvinyl alcohol fiber and acrylic acid, but is not limited thereto. Preferably, the substrate 110 includes the natural microfibers which do not include longitudinal fiber intertwining, so that the first surface 110a and/or the second surface 110b of the substrate 110 may be smoother and softer, being free from pilling. More preferably, the substrate 110 may include natural wood fiber material, which refers to a fiber material extracted from woody plant fiber without undergoing any processing, such as tencel cotton and the like, with the natural wood fiber material obtaining a relative smaller friction coefficient, and with a crystallinity of the natural wood fiber material being about 30%-70%. Accordingly, the frictional factor-static of the substrate 110 may be less than about 0.8, and the frictional factor-dynamic may be less than about 0.7, so that the substrate 110 may not be easily adhesive to wound openings, thereby prolonging the usage time of the wound dressing 100.

On the other hand, if the substrate 110 includes the polymer material for example a hydrophilic cross-linked polymer or a hydrophobic cross-linked polymer, wherein the hydrophilic cross-linked polymer may be selected from a group including gelatin, collagen, hyaluronic acid, alginate and chitosan, and the hydrophobic cross-linked polymer may be selected from a group including polyvinyl alcohol, polyoxyethylene, polyacrylonitrile, polystyrene, polyethylene, polypropylene, polymethylmethacrylate, polycarbonate, polyamide, polyurethane, olefin, vulcanizate and polyester.

Preferably, the substrate 110 may include the hydrophilic cross-linked polymer such as chitosan, for quickly absorbing blood and tissue fluid, wherein the de-vinylation degree of chitosan is about greater than 70%, the molecular weight is between about 10 kilodalton (KDa) and 1 megadalton (MDa), and the content of chitosan is about 10-1000 mg per 100 in per square centimeters of the substrate 110 (namely being about 50-150 mg/100 cm²), but not limited thereto. Accordingly, the wound dressing 100 may be allowable to keep the wound moist and to avoid wound frictional, so as to be applied on any kind of wounds.

With the aforementioned arrangements, the wound dressing 100 of the present embodiment may include both flexibility and antibacterial property, with the ductility of the wound dressing 100 being greater than 25% and with the breaking strength thereof being greater than 35 newton (N), so as to maintain the integrity and the fitness of wound dressing 100 in use. As an example, the aforementioned ductility and the breaking strength of the wound dressing 100 is analyzed for example by clamping the wound dressing 100 in a tensile testing machine with a clamping distance of 75 mm and a tensile rate of 300 mm/min until the wound dressing 100 breaks. Accordingly, the wound dressing 100 may therefore gain better antibacterial effect through the metal particles 130 embedded in the substrate 110. The metal particles 130 are not easily released from the substrate 110, to keep the wound from directly in contact with the metal particles 130, so as to avoid the issue of causing biological toxicity. In addition, the substrate 110 of the wound dressing 100 is soft and has better liquid absorption and air permeability, so that, a moisture vapor transmission rate of the wound dressing 100 may be about 4,000-20,000 g/m²*24 hr. The moisture vapor transmission rate refers to the amount of water vapor per unit (g) penetrate through per unit area (m²) of the wound dressing 100 during a period of time (24 hours) is about 4,000-20,000 g, but is not limited thereto. Accordingly, the wound dressing 100 may be applied on various wounds for a long time, for example being about 7-10 days to avoid excessive friction or adhesion.

People well known in the arts should easily realize the wound dressing of the present invention may further include other examples or varieties so as to meet the practical product requirements. The following description will detail the different embodiments of the wound dressing in the present invention. To simplify the description, the following description will detail the dissimilarities among the different embodiments and the identical features will not be redundantly described. In order to compare the differences between the embodiments easily, the identical components in each of the following embodiments are marked with identical symbols.

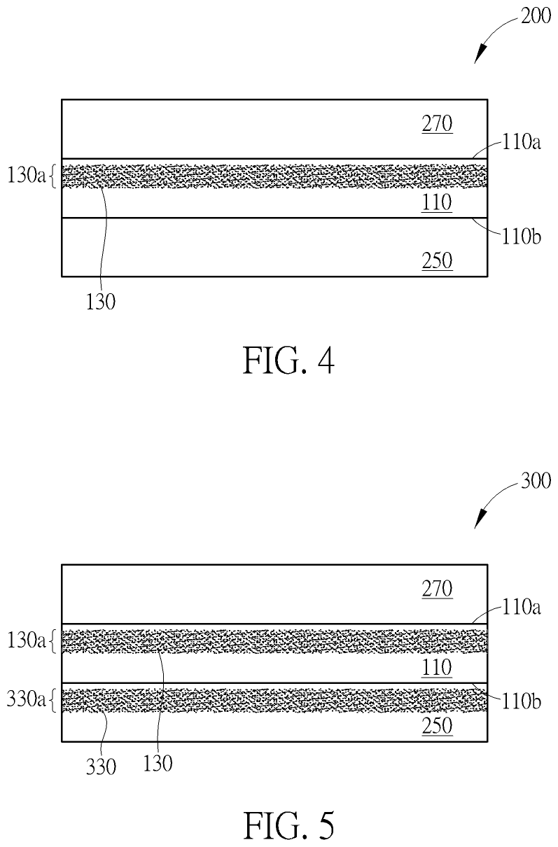

Please refer to FIG. 4, which illustrates a cross-sectional view of a wound dressing 200 according to the second embodiment of the present invention. In order to clearly show the detailed arrangement of each film of the wound dressing 200, the microstructures of the substrate 110 or other layers are omitted in FIG. 4. The structure and material of the wound dressing 200 in the present embodiment are substantially the same as those of the wound dressing 100 in the first embodiment, and the similarities will not be redundantly described hereinafter. The differences between the present embodiment and the aforementioned embodiments are in that the wound dressing 200 further includes an additional layer 250 which may include a fiber material or a polymer material to provide additional support or fixation for the substrate 110, or to provide additional liquid absorption, air permeability, anti-adhesion and the like for the substrate 110.

Precisely, the additional layer 250 may be disposed on the first surface 110a or the second surface 110b of the substrate 110, and the material selection thereof may be differ by the material of the substrate 110. In the embodiment that the substrate 110 includes the fiber material, the additional layer 250 preferably includes a hydrophilic cross-linked polymer layer or a hydrophobic cross-linked polymer layer, for achieving better liquid absorption and air permeability, such that, the substrate 110 may be more skin-friendly, so as to provide better wound caring. In one embodiment, the hydrophilic cross-linked polymer layer may be selected from a group of gelatin, collagen, hyaluronic acid, alginic acid and chitosan, and the hydrophobic cross-linked polymer layer may be selected from a group of polyvinyl alcohol, polyoxyethylene, polyacrylonitrile, polystyrene, polyethylene, polypropylene, polymethylmethacrylate, polycarbonate, polyamide, polyurethane, olefin, vulcanizate and polyester, but is not limited to. Otherwise, in another embodiment that the substrate 110 includes the polymer material, the additional layer 250 preferably includes a natural microfiber or a synthetic microfiber, to serve as a fixing layer for assisting the attachment or fixation of the substrate 110. In this way, the wound dressing 200 may provide better functions and advantages. The natural microfiber may be selected from a group of cotton, wood, linen fiber, jute fiber, ramie fiber, shengma fiber, hemp fiber, rayon, modal, lyocell and tencel cotton; and the synthetic microfiber may be selected from a group of natural or (semi) synthetic hydrophilic shortfibers including polyester (PET), polyamide 6 (PA6), polyamide 66 (PA66), nylon 6 (nylon 6), nylon 66 (nylon 66), polypropylene (PP), polyolefin, acetate fiber, polyethylene fiber, polyvinyl alcohol fiber and acrylic acid, but is not limited thereto.

Furthermore, the wound dressing 200 may additionally include a water-retaining layer 270, which may also be disposed on the first surface 110a or the second surface 110b of the substrate 110 to promote the wettability of the wound dressing 200. In this way, the water absorption of the wound dressing 200 may reach about 8 to 20 times more than the weight of the substrate 110, but is not limited thereto. In the present embodiment, the water-retaining layer 270 and the additional layer 250 are respectively disposed on the first surface 110a and the second surface 110b of the substrate 110, as shown in FIG. 4. However, people in the art should fully understand that the aforementioned arrangements of the water-retaining layer 270 and the additional layer 250 are only for examples, and which may include other suitable arrangements based on practical product requirements. In other embodiment, the water-retaining layer and the additional layer may also be sequentially stacked on the first surface 110a or the second surface 110b of the substrate 110, or the water-retaining layer or the additional layer may be optionally omitted.

With these arrangements, the wound dressing 200 of the present embodiment may include both the flexibility and the antibacterial property, and also to achieve better antibacterial effect and to reduce biological toxicity by disposing the metal particles 130 embedded in the substrate 110. In addition, the wound dressing 200 of the present invention further includes the additional layer 250 and/or the water-retaining layer 270, so as to further improve the functions and the stability of the wound dressing 200, with the moisture vapor transmission rate of the wound dressing 200 being about 4,000-20,000 g/m²*24 hr, and with the moisture vapor transmission rate referring to the amount of water vapor per unit (g) penetrating through per unit area (m²) of the wound dressing 200 during a period of time (24 hours). In other words, the wound dressing 200 has good moisture vapor transmission rate, and which is beneficial to be used on various wounds for a long tome such as 7-10 days, to avoid excessive friction or adhesion to wounds.

Please refers to Table 1 and Table 2 below, which respectively list the results of antibacterial property test and cytotoxicity test of various samples, wherein these samples include natural microfibers (for example including the tencel cotton), other wound dressings (for example including the tencel cotton and a chitosan layers stacked on one over another), the wound dressing 200 of the present invention with the substrate 110 of the wound dressing 200 including natural microfibers such as the tencel cotton, with the additional layer 250 of the wound dressing 200 including a hydrophilic cross-linked polymer layer such as chitosan, and with the metal particles 130 of the wound dressing 200 including silver particles being about 160 ppm in the substrate 110, and the wound dressing 100 of the present invention with the substrate 110 of the wound dressing 200 including natural microfibers such as the tencel cotton, and with the metal particles 130 of the wound dressing 200 including silver particles being about 160 ppm in the substrate 110, and a commercial wound dressing such as Anticoat. In the antibacterial property test, each sample is cut into a size with 0.4 g in weight, and three items of each sample are prepared and sterilized through an UV irradiation for 20 minutes. Next, a predetermined number of bacteria such as Staphylococcus aureus ATCC6538 or Escherichia coli ATCC8739 is added to each sample, and then, 20 ml of sterile buffer such as phosphate buffered saline (PBS) is added for fully infiltrating the samples. Then, 1 ml of liquid is collected from each sample, followed by appropriately diluting the liquid collected from each sample and coating on a medium with the colony number of each medium being about 200 colony-forming unit (CFU). Each medium is cultured under 37° C. for three days. According to what is shown in Table 1 and Table 2, the wound dressing 100 and the wound dressing 200 of the present invention do achieve better antibacterial effect, and the biocompatibility evaluation value of the wound dressing 100 and the wound dressing 200 of the present invention is 1 or 0 referring to the international standard: ISO-10993-5, wherein the evaluation value of 0 indicates the cell survival rate being about 90% to 100%, and the evaluation value of 1 indicates the cell survival rate being about 50% to 80%.

TABLE 1
Antibacterial Property Test

| | Antibacterial Property (%) | |
|---|---|---|
| | Staphylococcus | Escherichia |
| Samples | aureus | coli |
| natural microfibers | 0 | 0 |
| other wound dressings | 99.9997 | 99.9999 |
| wound dressing 200 | 99.9974 | 99.9999 |
| wound dressing 100 | 99.9907 | 99.9999 |
| commercial wound | 99.9383 | 99.9149 |
| dressing | | |

TABLE 2
Cytotoxicity Test

| | | | Biocompatibility | |
|---|---|---|---|---|
| | Survival | | Evaluation Value | |
| Samples | (%) | STD | (ISO-10993-5) | |
| wound dressing | 80 | 6.79 | 1 | (low) |
| 200 | | | | |
| other wound | 87 | 11.64 | 1 | (low) |
| dressings | | | | |
| wound dressing | 91 | 6.54 | 0 | (good) |
| 100 | | | | |
| natural | 93 | 15.75 | 0 | (good) |
| microfibers | | | | |
| commercial wound | 82 | 10.26 | 1 | (low) |

| | | | |
|---|---|---|---|
| dressing | | | |
| control | 100 | 6.53 | — |
| negative control | 102 | 11.38 | — |
| (non-toxic) | | | |
| positive control | 2 | 0.60 | — |
| (toxic) | | | |

The test samples of positive control (toxic) and negative control (non-toxic) shown in Table 2 are prepared according to ISO-10993-5.

Please refer to FIG. 5, which illustrates a cross-sectional view of a wound dressing 300 according to the third embodiment of the present invention. In order to clearly show the detailed arrangement of each film of the wound dressing 300, the microstructures of the substrate 110 and the additional layer 250 are omitted in FIG. 5. The structure and material of the wound dressing 300 in the present embodiment are substantially the same as those of the wound dressing 200 in the second embodiment, and the similarities will not be redundantly described hereinafter. The differences between the present embodiment and the aforementioned embodiments are in that a plurality of antibacterial metal particles 330 may be further embedded in the additional layer 250, between two opposite surfaces of the additional layer 250, for example through the same process such as a sputtering process. It is noted that the detailed arrangement, as well as the weight ratio, of the metal particles 330 in the additional layer 250 are basically the same as those of the metal particles 130 in the substrate 110 described above, and will not be described in detail here.

In the present embodiment, a concentration region 330a of the metal particles 330 in the additional layer 250 is preferably adjacent to the two opposite surfaces of the additional layer 250, for example, being adjacent to the surface which is closer to the substrate 110. For example, when the additional layer 250 is disposed on the second surface 110b of the substrate 110, the concentration region 330a may be disposed adjacent to the second surface 110b, as shown in FIG. 5, but not limited thereto. In other embodiments, the metal particles may also be disposed at one tenth, one third or one half of the thickness of the additional layer as reference to the aforementioned arrangements of the metal particles 130 in the substrate 110, due to practical product requirements. In addition, in the present embodiment, the metal particles 330 may also be elected from the group of silver, iron, nickel, copper, chromium, manganese, gold, gallium, mercury, lead, aluminum, zinc, bismuth, tin and palladium, preferably being silver particles, but not limited thereto. In the present embodiment, the metal particles 330 may include the same or different material as that of the metal particles 130, preferably both including silver particles, but not limited thereto.

With these arrangements, the wound dressing 300 of the present embodiment may achieve better antibacterial effect by disposing the metal particles 130, 330 embedded in the substrate 110 and the additional layer 250. Also, the metal particles 330 are not easily released from the additional layer 250, and which will not directly contact the wound for reducing the biological toxicity. In this way, the wound dressing 300 may therefore obtain better functions and stability, which is beneficial to be used on various wounds for a long time such as about 7-10 days so as to avoid excessive friction or adhesion to wounds.

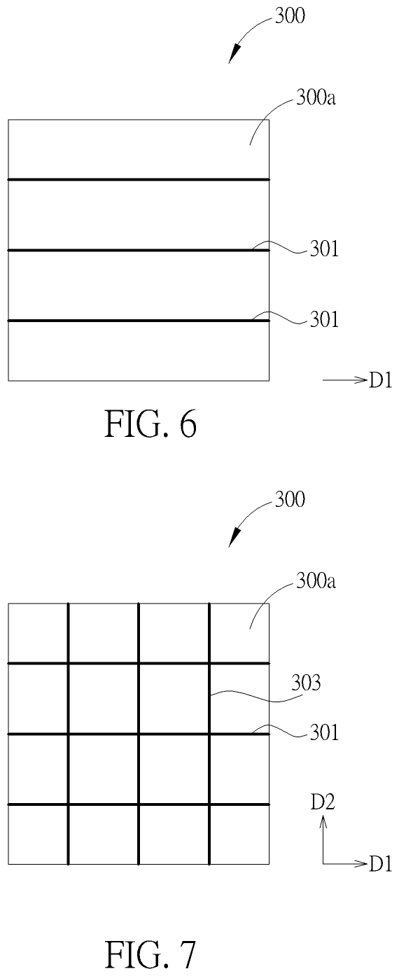

Please refers to FIG. 6, which illustrates a fixing structure of a wound dressing according to a preferably embodiment of the present invention. It is noted that while the wound dressing (such as the aforementioned wound dressing 300) includes a plurality films stacked from one over another, at least one fixing structure 301 may be further provided to fix the relative positions of the films. In the present embodiment, a plurality of fixing structures 301 may be optionally disposed on the wound dressing 300, and which may separately arranged from each other and extends along a direction (such as the x direction) D1, but is not limited thereto.

Precisely speaking, the fixing structure 301 may be any structure which is allowable to combine multiple film layers, such as a sewing line, a pressing line, an adhesive layer or the like. In the present embodiment, a sewing structure is disposed on a top surface 300a of the wound dressing 300 as an example, and however, the fixing structure of the present invention is not limited thereto. In addition, in another embodiment, the fixing structure may also be disposed on other part of the wound dressing 300, or extends along different directions. For example, as shown in FIG. 7, a plurality fixing structures 301 which is extended along the direction D1 and a plurality of fixing structure 303 which is extended along a direction D2 (such as the y-direction) are both disposed on the wound dressing 300, with the fixing structures 301 being interlaced with the fixing structures 303 to form a more steady structure, avoiding the possible separation or pealing of each film layers of the wound dressing 300. People in the art should fully understand that the aforementioned patterns of the fixing structures 301, 303 are only for example, and in another embodiment, the fixing structure may also be disposed into any suitable shapes or patterns such as a rectangular shape or triangle shape. Also, although the wound dressing 300 is exemplified in present embodiment, the aforementioned fixing structure may also be disposed on other wound dressings such as the wound dressing 100, 200 to prevent from the peeling or separation of the film layers thereof, due to practical product requirements.

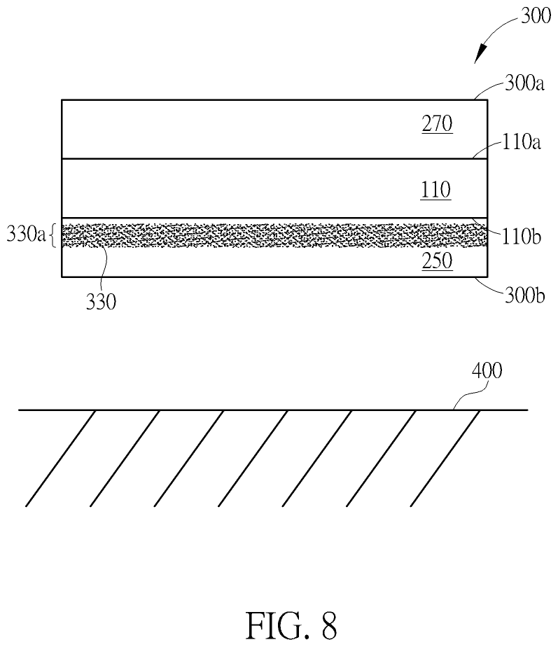

Please refers to FIG. 8, which illustrates the application of a wound dressing according to a preferably embodiment of the present invention. The wound dressing for example includes the additional layer 250, the substrate 110 and the water-retaining layer 270 stacked from bottom to top, to serve as the wound dressing 300, with the additional layer 250 preferably including hydrophilic cross-linked polymer such as chitosan, and with the substrate 110 preferably including a natural wood fiber material such as tencel cotton, but is not limited thereto. Accordingly, when the wound dressing 300 is applied to an injured part 400, the bottom surface 300b of the wound dressing 300 may directly cover the injured part 400 through the additional layer 250, so as to create a suitable healing environment, and also to avoid friction or adhesion to the injured part 400. It is noted that, the metal particles 130, 330 are respectively disposed in the substrate 110 and the additional layer 250, avoiding directly contacting the injured part 400. In this way, the wound dressing 300 may achieve better antibacterial effect and reduce biological toxicity through the metal particles 130, 330. Also, people in the art should fully understand that if the top surface 300a of the wound dressing 300 is used to cover the injured part 400 optionally, the metal particles 130, 300 may still free from direct in contact with the injured part 400.

Overall speaking, the present invention provides a wound dressing, in which a plurality of metal particles are embedded in the microstructures of the substrate, embedded between the gaps of the microstructures of the substrate, or embedded in the microstructures of both the substrate and the additional layer, with the metal particles being uncharged and difficult to release from the substrate or the additional layer. Accordingly, the wound dressing may achieve a better antibacterial effect with reduced biological toxicity. In addition, the substrate and/or the additional layer may respectively include a fiber material, preferably being a natural wood fiber such as tencel cotton, or a polymer material, preferably being a hydrophilic cross-linked polymer such as chitosan, so that, the wound dressing may be more skin-friendly, liquid absorption, air permeability, and the like. Through these arrangements, the wound dressing may be beneficial on absorbing tissue fluid and avoiding excessive friction or adhesion, effectively prolong the use time of the wound dressing, such as 7-10 days. Therefore, the wound dressing of the present invention may obtain better functionality and stability, so as to be applied on nursing various wounds.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A wound dressing, comprising:
a substrate, having a first surface and a second surface opposite to each other, the substrate comprising a plurality of microstructures;
a plurality of silver particles, embedded in gaps of the microstructures or embedded inside each of the microstructures, between the first surface and the second surface;
wherein the moisture vapor transmission rate of the wound dressing is 4000-20000 g/m²*24 hr.

2. The wound dressing accordingly to claim 1, wherein a concentration region of the silver particles is closed to the first surface or the second surface.

3. The wound dressing accordingly to claim 2, wherein the concentration region of the silver particles is disposed at one tenth of a thickness of the substrate.

4. The wound dressing accordingly to claim 2, wherein the concentration region of the silver particles is from one half of a thickness of the substrate to one tenth of the thickness of the substrate.

5. The wound dressing accordingly to claim 2, wherein the concentration region of the silver particles is from one third of a thickness of the substrate to one tenth of the thickness of the substrate.

6. The wound dressing accordingly to claim 1, wherein a concentration region of the silver particles extends from the first surface or the second surface of the substrate to one tenth of a thickness of the substrate.

7. The wound dressing accordingly to claim 1, wherein the microstructures comprise microfiber or microporous polymers.

8. The wound dressing accordingly to claim 7, wherein a length of each of the microstructures is 35-45 mm, and a density of the microstructures is 1.6-2.0 g/10 km.

9. The wound dressing accordingly to claim 7, wherein the microstructures comprise a natural microfiber or a synthetic microfiber.

10. The wound dressing accordingly to claim 9, wherein the natural microfiber is selected from a group of cotton, wool, linen fiber, jute fiber, ramie fiber, shengma fiber, hemp fiber, rayon, modal, lyocell fiber and tencel cotton.

11. The wound dressing accordingly to claim 9, wherein the synthetic microfiber is selected from a group of natural or (semi) synthetic hydrophilic shortfibers comprising polyester, polyamide 6, polyamide 66, nylon 6, nylon 66, polypropylene, polyolefin, acetate fiber, polyethylene fiber, polyvinyl alcohol fiber and acrylic acid.

12. The wound dressing accordingly to claim 9, further comprising:
a hydrophilic cross-linked polymer layer disposed on the first surface or the second surface of the substrate, wherein the hydrophilic cross-linked polymer layer comprises a material which is selected from a group of gelatin, collagen, hyaluronic acid, alginate acid and chitosan.

13. The wound dressing accordingly to claim 9, further comprising:
a hydrophobic cross-linked polymer layer disposed on the first surface or the second surface of the substrate, wherein the hydrophobic cross-linked polymer layer comprises a material which is selected from a group of polyvinyl alcohol, polyoxyethylene, polyacrylonitrile, polystyrene, polyethylene, polypropylene, polymethylmethacrylate, polycarbonate, polyamide, polyurethane, olefin, vulcanizate and polyester.

14. The wound dressing accordingly to claim 12, further comprising:
a water-retaining layer, disposed on the first surface or the second surface of the substrate, wherein the water-retaining layer and the hydrophilic cross-linked polymer layer are respectively disposed on the first surface and the second surface of the substrate.

15. The wound dressing accordingly to claim 13, further comprising:
a water-retaining layer, disposed on the first surface or the second surface of the substrate, wherein the water-retaining layer and the hydrophobic cross-linked polymer layer are respectively disposed on the first surface and the second surface of the substrate.

16. The wound dressing accordingly to claim 14, wherein the silver particles are also embedded in the hydrophilic cross-linked polymer layer, closed to a surface of the hydrophilic cross-linked polymer layer.

17. The wound dressing accordingly to claim 7, wherein the substrate comprises a hydrophilic cross-linked polymer layer or a hydrophobic cross-linked polymer layer.

18. The wound dressing accordingly to claim 17, further comprising:
a fixing layer, disposed on the first surface of the second surface of the substrate, the fixing layer comprises a natural microfiber or a synthetic microfiber, wherein the natural microfibers is selected from a group of cotton, wool, linen fiber, jute fiber, ramie fiber, shengma fiber, hemp fiber, rayon, modal, lyocell fiber and tencel cotton, and the synthetic microfiber is selected from a group of natural or (semi) synthetic hydrophilic shortfibers comprising polyester, polyamide 6, polyamide 66, nylon 6, nylon 66, polypropylene, polyolefin, acetate fiber, polyethylene fiber, polyvinyl alcohol fiber and acrylic acid.

19. The wound dressing accordingly to claim 17, wherein the hydrophilic cross-linked polymer layer comprises a material selected from a group of gelatin, collagen, hyaluronic acid, alginate and chitosan.

20. The wound dressing accordingly to claim 17, wherein the hydrophobic cross-linked polymer layer comprises a material selected from a group of polyvinyl alcohol, polyoxyethylene, polyacrylonitrile, polystyrene, polyethylene, polypropylene, polymethylmethacrylate, polycarbonate, polyamide, polyurethane, olefin, vulcanizate and polyester.

21. The wound dressing accordingly to claim 18, further comprising:
a water-retaining layer, disposed on the second surface or the first surface, the water-retaining layer and the fixing layer are separated disposed on the first surface or the second surface of the substrate.

22. The wound dressing accordingly to claim 17, wherein the silver particles are embedded in the fixing layer, closed to a surface of the fixing layer.

23. The wound dressing accordingly to claim 1, wherein a weight ratio of the metal particles related to the substrate is 10-250 mg metal particles in per 100 square centimeters of the substrate (10-250 mg/100 cm²).
